(19) United States
(12) Patent Application Publication
(10) Pub. No.: US 2006/0258755 A1
(43) Pub. Date: Nov. 16, 2006

(54) Preparation of sevoflurane with negligible water content

(76) Inventors: Ross C. Terrell, New Port Richey, FL (US); Joshua A. Levinson, Zionsville, PA (US); John C. McNeirney, Fairburn, GA (US)

Correspondence Address: HODGSON RUSS LLP, ONE M & T PLAZA, SUITE 2000, BUFFALO, NY, 14203-2391, US

(21) Appl. No.: 11/406,480

(22) Filed: Apr. 18, 2006

Related U.S. Application Data
(60) Provisional application No. 60/672,334, filed on Apr. 18, 2005.

(51) Int. Cl. A61K 31/075

(52) U.S. Cl. 514/715

(57) ABSTRACT

Provided is a sevoflurane anesthetic product which can remain substantially undegraded after long periods of storage, as well as a method for preparing the product. The product comprises sevoflurane in a glass container having a water content of less than 130 ppm. The method comprises drying sevoflurane having a water content of greater than 130 ppm to a water content les than 130 ppm. A preferred method of drying comprises contacting a sevoflurane composition having a water content of greater than 130 ppm with alumina-containing molecular sieves such that the water content is reduced to less than 130 ppm.

### CROSS REFERENCE TO RELATED APPLICATIONS

Applicants claim priority to Provisional Application No. 60/672,334, filed Apr. 18, 2005 and entitled “Preparation of Sevoflurane With Negligible Water Content,” which is incorporated herein by reference.

### FIELD OF THE INVENTION

The present invention is directed to the field of inhalation anesthetics, and more specifically, to the preparation of sevoflurane with negligible water content.

### BACKGROUND OF THE INVENTION

The compound sevoflurane (1,1,1,3,3,3-hexafluoroisopropyl fluoromethyl ether or (CF₃)₂CHOCH₂F) is a widely-used inhalation anesthetic, particularly suited for outpatient procedures. Economical and efficient methods for the preparation of stable sevoflurane are, therefore, highly desirable.

A number of methods for preparing sevoflurane have been described, many of which are of limited commercially viability. U.S. Pat. No. 3,683,092 describes four methods of preparation, three of which start with 1,1,1,3,3,3-hexafluoroisopropyl methyl ether (reacting with potassium fluoride in sulfolane or with bromine trifluoride) and one which starts with 1,1,1,3,3,3-hexafluoroisopropanol (reacting with formaldehyde and hydrogen fluoride). U.S. Pat. No. 3,897,502 describes the direct fluorination of 1,1,1,3,3,3-hexafluoroisopropyl methyl ether with elemental fluorine in argon. U.S. Pat. No. 4,874,901 discloses a halogen exchange reaction using sodium fluoride under supercritical conditions (i.e., high temperature and pressure). A fluorocarboxylation synthesis is reported in U.S. Pat. No. 4,996,371 utilizing bromine trifluoride. Bromine trifluoride is also used in an alternative synthesis described in U.S. Pat. No. 4,874,902. A further method of synthesis utilizing hexafluoroisopropanol, formaldehyde, hydrogen fluoride, and sulfuric acid is detailed in U.S. Pat. No. 4,250,334.

U.S. Pat. No. 5,969,193 prepares sevoflurane by an alternative process that is commercially viable. It provides 1,1,1,3,3,3-hexafluoroisopropyl methyl ether, chlorinates this material with chlorine to produce 1,1,1,3,3,3-hexafluoroisopropyl chloromethyl ether, and then fluorinates this intermediate in a mixture with hydrogen fluoride and a sterically-hindered amine to produce sevoflurane. U.S. Pat. No. 5,886,239 describes a similar synthetic method for producing sevoflurane using a different amine.

Currently available processes for the preparation of sevoflurane generally give a product containing dissolved water up to 0.12 to 0.14 wt %, or 1200 to 1400 ppm, the approximate water saturation limit of sevoflurane.

U.S. Pat. Nos. 5,990,176, 6,288,127, 6,444,859, and 6,677,492 (“Abbott patents”) indicate that the presence of water in sevoflurane is necessary in order that sevoflurane remain stable during storage in standard anesthetic packaging (e.g., Type III amber glass bottles, etc.). (As unsaturated sevoflurane is hygroscopic, solutions in storage often tend to increase in water content over time. The inclusion of water is thus particularly convenient).

Sevoflurane can undergo slight degradation during storage to produce, among other decomposition products, hydrofluoric acid, a well known glass etchant.

The Abbott patents teach that when sevoflurane is stored in glass bottles, the hydrofluoric acid so produced etches the inner surface of the bottle, exposing moieties, such as aluminum oxides, which act as Lewis acids, catalyzing additional sevoflurane degradation, by which process additional hydrofluoric acid is formed. As a result of accelerating production of hydrofluoric acid, a “cascade” of degradation takes place as the inner surface of the bottle becomes increasingly riddled with exposed Lewis acid moieties.

To address the problem of decomposition in sevoflurane during storage, it has been thought that Lewis acid inhibitors, such as, for example, water, should be present in stored sevoflurane solutions in order to prevent Lewis acid moieties in the etched glass from catalyzing a cascading degradation process. It is thought that a Lewis acid inhibitor must be present in an amount sufficient to prevent sevoflurane degradation in order to have a stable sevoflurane solution in the presence of Lewis acids such as etched glass. However, regulations require that the sevoflurane manufacturer demonstrate shelf life stability in the market package (e.g., glass, plastic, or metal). Thus, if the stability of sevoflurane can be demonstrated at low levels of dissolved water content, processes which leave low amounts of water in the sevoflurane product become useful methods of sevoflurane manufacture.

### BRIEF DESCRIPTION OF THE INVENTION

Surprisingly, it has been found that sevoflurane which is water-free and which is stored in standard glass anesthetic containers does not undergo degradation. Unlike the case of sevoflurane solutions having water content which is closer to saturation, sevoflurane compositions having lower amounts of water (at concentrations of less than 0.015 wt % or 150 ppm) can be stored in standard glass anesthetic containers without undergoing degradation. It has been found that sevoflurane solutions having low water contents in the range of from about 0.0 to 0.003 wt % (i.e., 0 to about 30 ppm) have long-term stability when stored in glass containers. The stability is seen even at temperatures in excess of room temperature. By stability is meant substantially undegraded as defined hereinafter and at a temperature of about 58° C. for a time of about 15 days. By “long term stability,” it is generally meant a stability of greater than two weeks and up to or even much longer than 24 months. Such stability can be observed in the absence of Lewis acid inhibitors of any type.

Thus, in one embodiment, the present invention provides stable sevoflurane having a water content of less than 150 ppm. In another embodiment, the invention provides stable sevoflurane solution having low water content. The water content range from approximately 8 to 30 ppm is hereafter referred to as “low” water content. In yet another embodiment, the invention provides a stable sevoflurane solution having negligible water content. The water content range from approximately 1 to 8 ppm is hereafter referred to as “negligible” water content. In yet another embodiment, the invention provides a stable sevoflurane solution which is essentially water-free, i.e., a water content of less than 1 ppm.

The sevoflurane is dried to negligible water levels as determined by standard water detection methods by removing its excess water via a drying process or agent (e.g., molecular sieves). It has also been discovered that the process of drying sevoflurane to low, “negligible” or “water-free” levels of water can be accomplished by the use of molecular sieves having Lewis acid properties, such as those comprised in part of aluminum oxides. The sevoflurane can actually be stored with the sieves for long periods of time without experiencing degradation. Thus, the stability of the solution is subsequently maintained, with no added water, in the presence of moieties, such as aluminum oxide, heretofore considered instrumental in the degradation of sevoflurane. By “water-free,” it is meant that the sevoflurane contains in the range of from 0 to 1 ppm of water as determined by Karl Fischer analysis.

Thus, in one embodiment, the present invention provides a method for drying sevoflurane to low, negligible or water-free water content. In one embodiment, the method comprises reducing the water level of a sevoflurane/water mixture by contacting it with a molecular sieve. In an additional embodiment, the contact takes place for long enough such that the water is reduced to negligible levels or below. In yet another embodiment, the sieves are stored with the sevoflurane for a period in excess of 30 days.

### DETAILED DESCRIPTION OF THE INVENTION

The present invention provides a stable, long-storing sevoflurane solution of low, negligible, or water-free water content.

Sevoflurane is primarily used as an inhaled anesthetic, and thus the solutions are generally relatively free of components, such as hydrofluoric acid and other breakdown products, such as, for example, 1,1,1,3,3,3-hexafluoroisopropanol, which are harmful if inhaled by humans. Otherwise, the stable sevoflurane solutions of the present invention can comprise other components in addition to water, such as other Lewis acids, for example. However, it is preferred that the sevoflurane have a purity of greater than 99.0 wt %. More preferred is a purity of greater than 99.90 wt %, and most preferred is a purity of greater than 99.97 wt %. The foregoing purities are calculated on a basis which does not include water.

An advantage of the compositions and methods of the present invention is that the sevoflurane solutions can be stable such that they remain substantially undegraded for long periods of time—30, 60, 90, 365 days, or longer, and often, effectively indefinitely. The solutions can exhibit the stability at temperatures as high as 40° C., and even as high as the boiling point of sevoflurane (58° C.) or higher. By “substantially undegraded,” it is meant that the degradant content of the solution is no more than 10,000 ppm. It is more preferred that the degradant content of the solution be no more than 3,000 ppm, and most preferred that the degradant content be no more than 300 ppm. The foregoing “ppm” measurements are calculated on a basis which does not include water.

The present invention also provides a method for preparing and maintaining the low or negligible water content of the sevoflurane solutions of the present invention. The stable sevoflurane compositions of the present invention can be prepared by subjecting a sevoflurane solution containing water to a drying process such as, for example, distillation, low temperature drying, potassium fluoride (KF), and molecular sieves. Sevoflurane can be commercially obtained and may also be prepared via one of many syntheses and preparations, several of which are described in various U.S. patents, such as U.S. Pat. No. 5,969,193, issued Oct. 19, 1999, the disclosure of which is hereby incorporated by reference.

A drying process or agent is used to achieve a water content in sevoflurane below about 0.013 wt % (or 130 ppm), preferably below 0.003 wt % (or 30 ppm), more preferably below 0.0008 wt % (or 8 ppm), and most preferably below 0.0001 wt % (or 1 ppm). Such processes or agents may include—but are not limited to—the use of molecular sieves, low temperature drying, potassium fluoride (KF), and distillation. If distillation is used, it may be necessary to distill for long periods to achieve the low water, negligible water or water-free sevoflurane solutions of the present invention.

Low temperature drying comprises lowering the water-containing sevoflurane solution to temperatures as low as −30° C. or lower such that ordered water molecule structures are formed. When using low temperature drying as the drying process, the sevoflurane should be cooled below the freezing point of water (i.e., 0° C.), preferably between −30° C. to −20° C. The water structures can be subsequently removed, such as by filtration with a stainless steel filter element. This is generally done after a low liquid temperature has been reached, and preferably held, for a period of time (e.g., 24 hours).

A preferred method for producing stable sevoflurane of negligible water content is the exposure of the solution to molecular sieves which are comprised, in part, of alumina. The use of alumina-containing sieves, which introduces a known Lewis acids due to the aluminum oxide content, surprisingly does not result in degradation of the sevoflurane, even after the solution has been dried to low, negligible, or water-free moisture content. The lack of degradation occurs even in cases such that the sieves render the solution essentially anhydrous and are subsequently stored with the solution for long periods of time.

The method comprises combining a sevoflurane solution comprising water at or below saturation levels with the molecular sieves such that the water level in the solution is lowered to 120 ppm or below. Preferably, the water level is lowered to low levels, i.e., 30 ppm or below, and more preferably, to negligible levels, i.e., 8 ppm or below.

In general, molecular sieves are comprised of a mixture of inorganic constituents to produce a desired porous structure that can selectively trap a target molecule. These constituents generally include primarily alumina (aluminum oxide) and amorphous silica with various proportions of sodium oxide, potassium oxide, calcium oxide, and binder material. The proportion and/or combination of these species determines the pore size, which is typically 2 Å or greater, with commonly available sieve sizes being 3, 4, 5, or 10 Å (angstroms).

Direct contact of the molecular sieves with sevoflurane can be performed at ambient conditions, preferably between 10° C. and 30° C. The amount of molecular sieve material to use should be sufficient to remove dissolved water to the desired level, preferably between 1 wt % to 20 wt % of molecular sieves should be used relative to the weight of sevoflurane. The composition of the molecular sieves which can be used in the process of the present invention are preferably comprised in part of alumina. They are more preferably comprised of alumina in amounts in the range of 25 to 50 wt %. The sieves generally have cavity sizes in the range of from 2 to 12 Å, and more preferably in the range of from 2 to 5 Å. Most preferred are sieves with a cavity size of about 3 angstroms (i.e., nominal pore size of 3 Å) such as Type 3A, although other pore sizes could be used with varying degrees of removal. The sieves and the sevoflurane solution are preferably contacted in amounts and for times that render the water content of the sevoflurane to less than 30 ppm. Under fixed-bed flow conditions, this may correspond to 10 minutes or more of contact. Under stirred conditions, this may correspond to 30 minutes or more of contact. Under stationary conditions, this may correspond to 3 hours or more of contact.

Other methods of drying, can be used to dry the sevoflurane to low, negligible or water-free water levels. When using potassium fluoride (KF) as the drying agent, direct contact of the KF with sevoflurane can be performed at ambient conditions, preferably between 10° C. and 30° C. The amount of KF to use should be sufficient to remove dissolved water to the desired level, preferably 2 wt % to 20 wt % of KF should be used relative to the weight of sevoflurane. Solid material can be removed via filtration (e.g., a stainless steel filter or a polymer fiber filter) after achieving the desired water concentration.

The contact time between sevoflurane and any drying process or drying agent used should be sufficient to remove the dissolved water to the desired level. Stirring or another form of agitation within the knowledge of one skilled in the art may be used to facilitate water removal. The sevoflurane and the employed drying process or agent may be separated, if desired, at completion of drying. Methods of separation, such as mechanical separation, for example, are within the knowledge of one skilled in the art.

It should be understood that included in the ambit of the present invention are stable sevoflurane solutions having low, negligible or water-free water levels regardless of whether or how the solution was subjected to a drying process. The low water, negligible water or water-free solutions of the present invention are generally free of degradation regardless of whether they have been subjected to a drying step or how they were dried.

The sevoflurane solutions of the present invention can be shipped and/or stored in a wide variety of containers without undergoing degradation. Suitable containers include those of glass, polyethylene, stainless steel, as well as containers having linings that that are inert to sevoflurane, such as, for example, epoxy-phenolic lining. Especially convenient and preferred are glass containers, particularly containers made of Type III amber glass.

The present invention demonstrates that the low-water sevoflurane solutions described herein can be stored in glass containers containing identified Lewis acids (e.g., alumina). Surprisingly, the low-water sevoflurane solutions of the present invention are stable in the presence of aluminum oxide moieties (a Lewis acid moiety), and thus the solutions are generally stable in the presence of glass having such moieties.

The stable sevoflurane compositions of the present invention have a water content of less than 130 ppm. In another embodiment, the water content is less than 80 ppm. In another embodiment, the water content is less than 30 ppm. In a preferred embodiment, the water content is in the range of from 0 to 8 ppm. The foregoing water contents are based upon the combined weight of the sevoflurane and water. The water content can be measured by standard detection methods—e.g., by Karl Fischer. The water content should be at or below about 0.015 wt % (or 150 ppm), preferably below 0.003 wt % (or 30 ppm), and more preferably below 0.0008 wt % (or 8 ppm).

The solutions of the present invention are generally expected to free of degradation if shipped and stored in standard anesthetic containers. In particular, shipping and storage of the solutions in containers of glass generally will not result in degradation of the sevoflurane. In fact, sevoflurane which has been stored for as long as 30, 60, 90, or even 365 days in glass bottles can be greater than 99 wt % pure, and even as high as or higher than 99.99 wt % pure.

The examples appearing below and in the discussion above help to fully illustrate the practice of prepared embodiments of the present invention. These examples are intended to be for illustrative purposes only and are not intended to limit the scope of the invention.

### EXAMPLE 1

The following experiment demonstrates the preparation of low water sevoflurane and its subsequent stability. In one experiment initiated in August 2000, sevoflurane (Abbott Laboratories, Lot #61-339-DK, Expiration Date Aug. 1, 2001) was dried to 0 ppm water content using Type 3A molecular sieve. Drying was performed by mixing the sevoflurane with molecular sieves and then allowing these materials to stand together for several hours. The water content was determined by Karl Fischer analysis. A sample of the dried sevoflurane was placed in a new amber Type III glass bottle, which had been dried at 100° C. for 2 hours. The bottle was sealed with a black phenolic/urea resin cap and a polyseal liner of polyethylene resin and shrink-wrapped or wrapped with Teflon® tape and shrink-wrapped. The sample was then held at room temperature (25-27° C.) and at ambient relative humidity. At the end of the four-week stability run, the sample was analyzed for % water (Karl Fischer analysis) and for sevoflurane purity by gas chromatography, and it was found to have 68 ppm water and to be 99.998% sevoflurane; there was no decomposition.

### EXAMPLE 2

One undried lot of sevoflurane from Abbott Laboratories (Lot #0 335 70 K, Expiration Date Apr. 1, 1997, stored in a Type III bottle) was analyzed in May and October 2000. This sevoflurane was 97 ppm H₂O and 99.9916% sevoflurane by gas chromatography. The expiration date on this sample was 1997 with a shelf life of 2 years, indicating it was probably packaged in 1995 and, therefore, had been stored at ambient temperature for about five years at the time of this analysis without decomposition.

### EXAMPLE 3

Additional data related to the stability of sevoflurane was received from the U.S. Government, listing the % water of 71 lots of sevoflurane manufactured by Abbott Laboratories prior to Jan. 27, 1997. The water content of these lots ranged from 0.0008 to 0.0131 wt % (i.e., 8 ppm to 131 ppm). From.this list, a group of lots were recalled because of instability; this information was obtained from the FDA via the Freedom of Information Act. There were, in fact, 19 of the 71 lots recalled because of instability and/or decomposition. The water contents of the 1-9 recalled lots (average 0.0036% or 36 ppm) was similar to the water contents of the 52 lots not recalled (average 0.0036% or 36 ppm). The lots recalled are not evenly distributed among these lots, which were listed in chronological order. The majority of the unstable lots are grouped together in the later part of the series. Abbott surmised in related documents that the root cause of the degradation could have been rust (i.e., iron oxide, a Lewis acid), introduced into the sevoflurane from a rusty valve on a bulk container.

### EXAMPLE 4

This example demonstrates that degradation can occur in the presence of iron oxide (a Lewis acid) at low levels of dissolved water and that this degradation can be mitigated at higher water levels.

A sample of sevoflurane containing 30 ppm of water and 0.05 g Fe₂O₃ (iron oxide) was placed in a new Type III amber glass bottle in July 2000 and sealed as previously described. After four weeks at 40° C., gas chromatographic analyses showed 90.7% sevoflurane, 6.33% (CF₃)₂CHOCH₂OCH(CF₃)₂, and 0.49% (CF₃)₂CHOH, along with three other unidentified decomposition products.

To a second sample of sevoflurane (28 g) saturated with water (1235 ppm) was added 0.07 g Fe₂O₃. This sample was sealed in September 2000 in a new Type III glass bottle as previously described and stored at 40° C. for four weeks. There was no decomposition of the sevoflurane observed, which was determined by gas chromatography to be 99.97% pure.

In light of this example, it is even more surprising that degradation is not observed for low water content sevoflurane stored in glass containers and/or contacted and/or stored with molecular sieves that are comprised, at least in part, of alumina.

### EXAMPLE 5

Another sample of sevoflurane (40 g) was stored with 2 g of Type 3A molecular sieve (an alumino silicate that contains Al₂O₃, identified as a Lewis acid in the '176 patent) in July 2000. This sample was in a new Type III glass container for six months at ambient temperatures. No decomposition of the sevoflurane was observed as determined by gas chromatography indicating greater than 99.99% sevoflurane.

### EXAMPLE 6

Starting in July 2000, 40 g of sevoflurane (Abbott Laboratories Lot # 35-621 -DK-03) was stored in the presence of 2 g of Type 3A molecular sieve for 20 months in a dried, new Type III amber glass bottle at room temperature. The initial water content was 218 ppm and was essentially zero after 16 hours of contact with the molecular sieve (as determined by Karl Fischer, a method known to those skilled in the art with a detection limit of about 1 ppm or less). At the end of the 20 month trial, there was no evidence of any decomposition as shown by gas chromatography at 99.997% sevoflurane.

### EXAMPLE 7

Starting in July 2000, a sample of sevoflurane (from the lot described in Example 1) was held at room temperature (25-27° C.) over Type 3A molecular sieve for two-and-a-half months in a dried, new Type III amber glass bottle. At the end of this time, the % sevoflurane was greater than 99.99% (as determined by gas chromatography). There was no decomposition.

### EXAMPLE 8

Sevoflurane (99.99%, produced January 2005) was dried over Type 3A molecular sieve using a continuous flow bed apparatus to a water-composition of 0.0 wt % (or 0 ppm) as measured by Karl Fischer. 100 ml of sevoflurane was packaged into a Type III amber glass bottle and sealed for a 30-day stability trial at 40° C. and 75% relative humidity. At the end of this time, the sevoflurane was 99.99% as determined by gas chromatography. There was no decomposition.

### EXAMPLE 9

Sevoflurane (99.99%, produced January 2005) was dried over Type 3A molecular sieve using a continuous flow bed apparatus to a water composition of 0.0 wt % (or 0 ppm) as measured by Karl Fischer. 250 ml of sevoflurane was packaged into a Type III amber glass bottle and sealed for a 30-day stability trial at 40° C. and 75% relative humidity. At the end of this time, the sevoflurane was 99.99% as determined by gas chromatography. There was no decomposition.

### EXAMPLE 10

Sevoflurane (99.99%, produced January 2005) was dried over Type 3A molecular sieve using a continuous flow bed apparatus to a water composition of 0.0 wt % (or 0 ppm) as measured by Karl Fischer. 28.1 kg of sevoflurane was packaged in a five-gallon epoxy-lined drum and sealed for a 30-day stability trial at 40° C. and 75% relative humidity. At the end of this time, the % sevoflurane was 99.99% as determined by gas chromatography. There was no decomposition.

### EXAMPLE 11

Additional data demonstrating the long-term stability of Sevoflurane are presented in the following table. Purified Sevoflurane was dried over Type 3A molecular sieve using a continuous flow bed apparatus to negligible or near-negligible water compositions as measured by Karl Fischer. The dried material was packaged into 100 mL or 250 mL Type III amber glass bottles or into 5 gal epoxy-lined drums and subsequently stored as part of a stability trial under controlled or ambient conditions. In all cases, the % Sevoflurane—as determined by gas chromatography—indicated no decomposition.

| Container | Mfg. | Initial | | | Final | |
|---|---|---|---|---|---|---|
| Type | Date | Water | Duration | Conditions | Comp. | Result |
| 100 mL | February | 0 ppm | 60 days | 40 C./75% | 99.99% | No |
| | 2005 | | | RH | | decomposition |
| | February | 0 ppm | 9 months | 25 C./60% | 99.98% | No |
| | 2005 | | | RH | | decomposition |
| 250 mL | February | 0 ppm | 60 days | 40 C./75% | 99.98% | No |
| | 2005 | | | RH | | decomposition |
| | April | 5 ppm | 90 days | 40 C./75% | 99.997% | No |
| | 2005 | | | RH | | decomposition |
| | January | 0 ppm | 12 months | 25 C./60% | 99.99% | No |
| | 2005 | | | RH | | decomposition |
| | February | 0 ppm | 9 months | 25 C./60% | 99.98% | No |
| | 2005 | | | RH | | decomposition |
| | April | 5 ppm | 6 months | 25 C./60% | 99.997% | No |
| | 2005 | | | RH | | decomposition |
| 5 gal | January | 0 ppm | 6 months | 40 C./75% | 99.98% | No |
| | 2005 | | | RH | | decomposition |
| | February | 0 ppm | 12 months | 40 C./75% | 99.98% | No |
| | 2005 | | | RH | | decomposition |
| | April | 6 ppm | 60 days | 40 C./75% | 99.998% | No |
| | 2005 | | | RH | | decomposition |
| | January | 0 ppm | 90 days | Ambient | 99.99% | No |
| | 2005 | | | | | decomposition |
| | February | 0 ppm | 12 months | Ambient | 99.98% | No |
| | 2005 | | | | | decomposition |
| | April | 10 ppm | 9 months | Ambient | 99.998% | No |
| | 2005 | | | | | decomposition |

The sevoflurane used in examples 8-11 was produced by the method described in pending U.S. patent application Ser. No. 11/281,293, filed Nov. 17, 2005, the disclosure of which is incorporated herein by reference.

While embodiments of the invention have been described in detail, that is done for the purpose of illustration, not limitation.

1. An anesthetic product comprising:
a) a glass container having an inner wall which defines a space; and
b) a volume of a composition comprising sevoflurane and, optionally, water, and contained in said space such that the composition contacts said inner wall; wherein the water is present in the composition in amounts of less than 130 ppm, and wherein the composition is capable of remaining substantially undegraded in the container for time periods in excess of 365 days at temperatures below 58° C.

2. A product as in claim 1 wherein the water is present in amounts of less than 30 ppm.

3. A product as in claim 1 wherein the water is present in amounts of less than 8 ppm.

4. A product as in claim 1 wherein the composition is essentially water-free.

5. A product as in claim 1 wherein the glass container is a Type III amber glass container.

6. A product as in claim 1 wherein the glass container is new.

7. A product as in claim 1 wherein the composition is capable of remaining substantially undegraded in the container for time periods in excess of 15 days at temperatures below 45° C.

8. A product as in claim 1 herein the glass container has not previously been used to store sevoflurane.

9. An anesthetic product comprising
a) a glass container having an inner wall which defines a space; and
b) a volume of a composition comprising sevoflurane and, optionally, water, and contained in said space such that the composition contacts said inner wall; wherein the water is present in the composition in amounts of less than 130 ppm, and wherein the composition has been stored in the glass container for a time period in excess of 365 days at temperatures below 58° C.

10. A product as in claim 9 wherein the water is present in amounts of less than 30 ppm.

11. A product as in claim 9 wherein the water is present in amounts of less than 8 ppm.

12. A product as in claim 9 wherein the composition is essentially water-free.

13. A product as in claim 9 wherein the glass container is a Type III amber glass container.

14. A product as in claim 9 wherein the glass container is new.

15. A product as in claim 9 herein the glass container has not previously been used to store sevoflurane.

16. A method for reducing the water content of a sevoflurane solution, said method comprising the following steps:
a) providing a solution comprising sevoflurane and water; wherein the solution has a water content in excess of 130 ppm.
b) drying said solution such that the water content of the solution is less than 130 ppm based upon the combined weight of the sevoflurane and water.

17. A method as in claim 16 wherein step b) comprises the use of one or more methods selected from the group consisting of: molecular sieves, low temperature drying, distillation and contact with potassium fluoride.

18. A method as in claim 16 wherein step b) comprises contacting the solution with a molecular sieve.

19. A method as in claim 18 wherein the sieve comprises one or more aluminum oxides.

20. A method as in claim 18 wherein the sieve content is in the range of 1 to 20 wt %, based upon the combined weight of the solution and the sieves.

21. A method as in claim 18 wherein the sieve and solution are contacted for a time in the range of from 0.5 to 20 days.

22. A method as in claim 18 wherein the solution is contacted with the sieve until the solution is water-free.

23. A method as in claim 16 wherein step b) comprises drying said solution such that the water content of the solution is less than 30 ppm based upon the combined weight of the sevoflurane and water.

24. A method as in claim 16 wherein step b) comprises drying said solution such that the water content of the solution is less than 8 ppm based upon the combined weight of the sevoflurane and water.

25. A method as in claim 16 wherein step b) comprises drying said solution such that the solution is essentially water free.
